(19) United States
(12) Patent Application Publication
(10) Pub. No.: US 2015/0126851 A1
(43) Pub. Date: May 7, 2015

(54) BIOPSY NEEDLE GUIDING APPARATUS FOR STEREOTACTIC BIOPSY, IMAGING APPARATUS HAVING THE SAME AND BIOPSY METHOD USING THE SAME

(71) Applicant: Rayence Co., Ltd., Gyeonggi-do (KR)

(72) Inventor: In Jae BAEK, Gyeonggi-do (KR)

(73) Assignee: Rayence Co., Ltd., Gyeonggi-do (KR)

(21) Appl. No.: 14/211,349

(22) Filed: Mar. 14, 2014

(30) Foreign Application Priority Data
Nov. 6, 2013 (KR) 10-2013-0134236

(51) Int. Cl. A61B 6/12; A61B 6/00; A61B 17/34; A61B 10/02

(52) U.S. Cl. CPC A61B 6/12; A61B 10/0266; A61B 6/4007; A61B 17/3403; A61B 6/502; A61B 2010/0208
USPC 600/424

(57) ABSTRACT

The present invention provides a biopsy needle guiding apparatus which makes it possible to precisely and rapidly perform stereotactic biopsy, an imaging apparatus having the same and a biopsy method using the same. The biopsy needle guiding apparatus includes a needle guiding apparatus body having a biopsy needle, and at least one X-ray source provided in the needle guiding apparatus body.

### BACKGROUND OF THE INVENTION

1. Field of the Invention

The present invention relates generally to biopsy needle guiding apparatuses, imaging apparatuses having the same and biopsy methods using the same, and more particularly, to a biopsy needle guiding apparatus which makes it possible to precisely and rapidly perform stereotactic biopsy, an imaging apparatus having the same and a biopsy method using the same.

2. Description of the Related Art

X-ray is a generic term for electromagnetic waves of short-wavelength corresponding to wavelengths in the range of 0.01 to 10 nanometers and frequencies in the range 30×10¹⁵ Hz to 30×10¹⁸ Hz. X-ray imaging is a kind of radiography which projects the interior of a target body using X-rays having high penetrating power. As is well known, X-rays involve an attenuation phenomenon, such as compton scattering, photoelectric effect, etc., depending on the quality, density and thickness of an object, when passing through the object. Therefore, in X-ray imaging, based on an attenuation rate of X-rays accumulated while passing through the target body, the interior of the target body is projected onto a plane by X-rays. For this, an exclusive X-ray system is used.

Recently, grafting onto technologies in the semiconductor field, X-ray imaging technology has continued to rapidly evolve, in lieu of typical analog methods using films, into digital X-ray image technology that has many advantages, e.g., comparatively high resolution, wide dynamic area, easy electrical signal generation, simple data processing and storage, etc. Digital-based imaging technology greatly reflects clinic environmental requirements such as an early diagnosis of disease on the basis of excellent diagnostic ability of a digital image.

In response to this, digital mammography, which is a breast X-ray imaging technology, was introduced. This technology can produce a high-resolution image showing the internal structure of a target body, e.g., the breast a patient, using the intrinsic biological tissue comparing ability of X-rays, thus making it possible to detect a lesion or micro-calcification for detection and an early diagnosis of breast cancer. Digital mammography is rapidly coming into wide use by virtue of not only many advantages of the digital X-ray imaging technology but also intrinsic characteristics, such as minimization of radiation exposure through reduction in the number of imaging procedures, image magnification, an increase in resolution, and adjustability of brightness and a contrast ratio.

Furthermore, a digital mammography apparatus which can immediately check an abnormal portion (lesion) of the breast of the patient via X-ray imaging is provided with a biopsy needle module for extracting the abnormal portion of the breast, thus making it possible to check the abnormal portion and extract tissue of the abnormal portion at the same time.

Referring to FIG. 1 and FIG. 2 respectively showing a conventional mammography apparatus and a main body thereof, the mammography apparatus 1 includes a support column 11 which has a vertical column shape, and a C-arm or main body 10 which is provided so as to be movable upwards or downwards along the support column 11 and has a general C shape or the like when viewed along the x-axis. A generator or an X-ray source 20 which emits X-rays towards a lower end of the main body 10 is provided on an upper end of the main body 10. Facing the X-ray source 20, a detector 50 is provided on the lower end of the main body 10.

The X-ray source 20 is installed in the main body 10 so as to be rotatable on the y-axis by an X-ray source rotating support 22. Fixed to the main body 10, a needle guiding apparatus holder 30 is installed between the X-ray source 20 and the detector 50. A biopsy needle guiding apparatus 40 is installed on the needle guiding apparatus holder 30. Here, the biopsy needle guiding apparatus 40 is provided on the needle guiding apparatus holder 30 so as to be movable in x-, y- and z-axis directions.

In the mammography apparatus 1 having the above-mentioned construction, when a patient is positioned in an imaging position standing up or sitting down, the main body 110 is moved upwards or downwards along the support column 11 and adjusted in height such that the target body (BR of FIG. 2), for example, the breast, of the patient is placed at a desired position on the detector 150. Thereafter, the X-ray source 20 emits X-rays to the target body BR. The detector 150 which is disposed under the target body BR receives the X-rays that have been passed through the target body BR and obtains an image.

Subsequently, as shown in FIG. 2, the X-ray source rotating support 22 is rotated to positions indicated by the solid line and dotted line, for example, to the left and the right each by an angle ranging from 15 degrees to 30 degrees. X-ray imaging of the target body BR placed on the detector 50 is additionally performed at each of the left and right positions.

Using trigonometry from the obtained three X-ray images of the target body BR, (if an abnormal portion is present in the target body BR) a three-dimensional position, that is, x-, y- and z-positions, of the abnormal portion can be calculated.

Thereafter, based on the calculated three-dimensional position of the abnormal portion, the needle guiding apparatus holder 30 moves the biopsy needle guiding apparatus 40 to a corresponding position, and a needle 44 provided on the biopsy needle guiding apparatus 40 is moved forwards and backwards to extract tissue of the abnormal portion of the target body BR.

However, in the conventional mammography apparatus having the above-mentioned construction, a rotating drive unit for rotating the X-ray source 20 by predetermined angles is required to image the target body BR at three different angles. The rotating drive unit makes the overall structure of the mammography apparatus complex and also increases the size thereof, thus increasing the production cost.

Furthermore, to calculate a three-dimensional position of a target from images obtained at three different angles using the rotating drive unit, the angle by which the X-ray source 20 is rotated must be accurate. Even after the X-ray source has rotated, X-ray imaging must be performed in a completely stationary state. Therefore, it is difficult to accurately control the rotation angle of the X-ray source 20.

Particularly, a need for the rotation of the X-ray source 20 using the rotating drive unit when imaging the target body BR increases the time it takes to image. Moreover, while imaging and extracting the tissue of the abnormal portion, the target body BR must be maintained in the stationary state. This causes the patient and an inspector to experience much fatigue.

If the target body BR moves while imaging the target body BR, it is very difficult to obtain a correct three-dimensional position of the abnormal portion. Even though the correct three-dimensional position is obtained, if the target body BR moves while the biopsy needle guiding apparatus 40 moves to the corresponding position, it may not be able to accurately extract the tissue of the desired abnormal portion.

### SUMMARY OF THE INVENTION

Accordingly, the present invention has been made keeping in mind the above problems occurring in the prior art, and an object of the present invention is to provide a biopsy needle guiding apparatus which makes it possible to precisely and rapidly perform stereotactic biopsy, an imaging apparatus having the same and a biopsy method using the same.

In order to accomplish the above object, in an aspect, the present invention provides a biopsy needle guiding apparatus, including a needle guiding apparatus body provided with a biopsy needle, and at least one X-ray source provided on the needle guiding apparatus body.

The at least one X-ray source may comprise one X-ray source provided so as to be movable in a direction crossing a longitudinal direction of the biopsy needle.

The at least one X-ray source may comprise two or more X-ray sources spaced apart from each other in a direction crossing a longitudinal direction of the biopsy needle.

The X-ray sources may be arranged at left and right symmetrical positions spaced apart from a point aligned with a longitudinal axis of the biopsy needle by a same distance within an angular range from 15 degrees to 30 degrees.

The biopsy needle guiding apparatus may further include an X-ray source disposed at a position aligned with the longitudinal axis of the biopsy needle.

In another aspect, the present invention provides an imaging apparatus, including a main X-ray source emitting X-rays to a target body, a detector receiving the X-rays that have passed through the target body, a main body supporting the X-ray source and the detector, and a biopsy needle guiding apparatus having a needle guiding apparatus body provided on the main body between the main X-ray source and the detector, the needle guiding apparatus body being provided with a biopsy needle, and at least one X-ray source provided on the needle guiding apparatus body.

The biopsy needle guiding apparatus body may be removably provided on the main body.

In a further aspect, the present invention provides a biopsy method using an imaging apparatus including a main X-ray source and a detector disposed facing each other with a target body therebetween, and a biopsy needle guiding apparatus disposed between the target body and the main X-ray source, the biopsy needle guiding apparatus comprising a biopsy needle and at least one X-ray source, the biopsy method including obtaining an X-ray transmission image of the target body and determining whether the target body is normal or abnormal, and calculating a three-dimensional position of an abnormal portion of the target body using the needle X-ray source and the detector, and extracting tissue of the abnormal portion using the biopsy needle.

A biopsy needle guiding apparatus, an imaging apparatus having the same and a biopsy method using the same according to the present invention make it possible to rapidly and accurately detect a three-dimensional position of an abnormal portion and extract tissue of the abnormal portion, thus reducing the time it takes to perform X-ray imaging and extract the tissue of the abnormal portion.

Furthermore, a mammography apparatus, that is, the imaging apparatus, according to the present invention has a simple structure having no separate drive unit, thus facilitating maintenance of the apparatus, extending the lifetime of the apparatus, and reducing the size thereof. For this reason, the production cost of the imaging apparatus can be reduced.

In accordance with the biopsy needle guiding apparatus, the imaging apparatus having the same and the biopsy method using the same according to the present invention, because the abnormal portion can be rapidly and accurately detected and the tissue thereof can also be rapidly and accurately extracted, the fatigue degree of a patient and inspector can be reduced.

Moreover, if the biopsy needle guiding apparatus according to the present invention is installed in the existing mammography apparatus which has not been able to conduct the stereotactic biopsy, it becomes possible to perform stereotactic biopsy even using an existing mammography apparatus. As such, the applicability of the existing apparatus can be enhanced by the present invention.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a front view showing a mammography apparatus according to the conventional art;

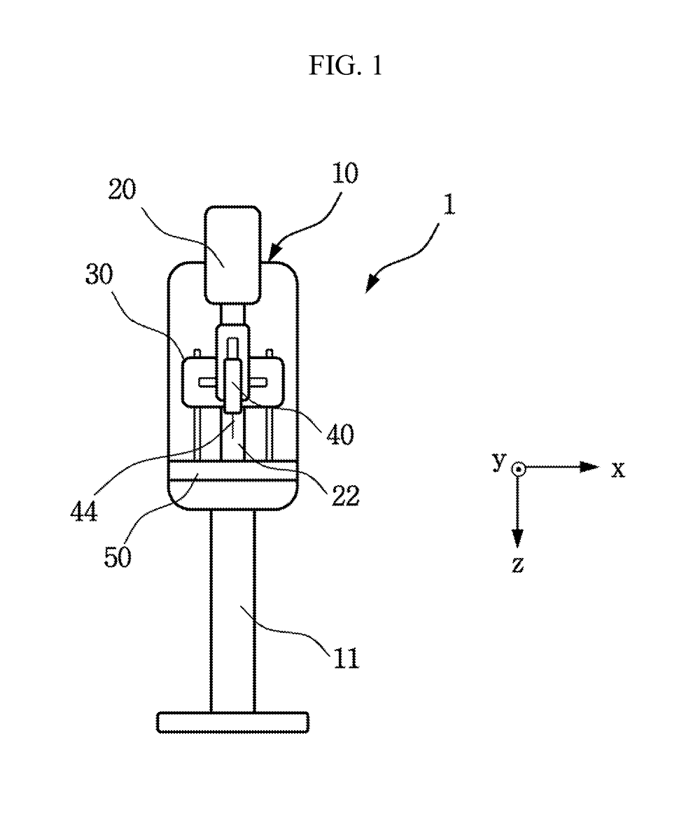

FIG. 2 is a front view showing a main body of the mammography apparatus so as to illustrate a stereotactic biopsy process;

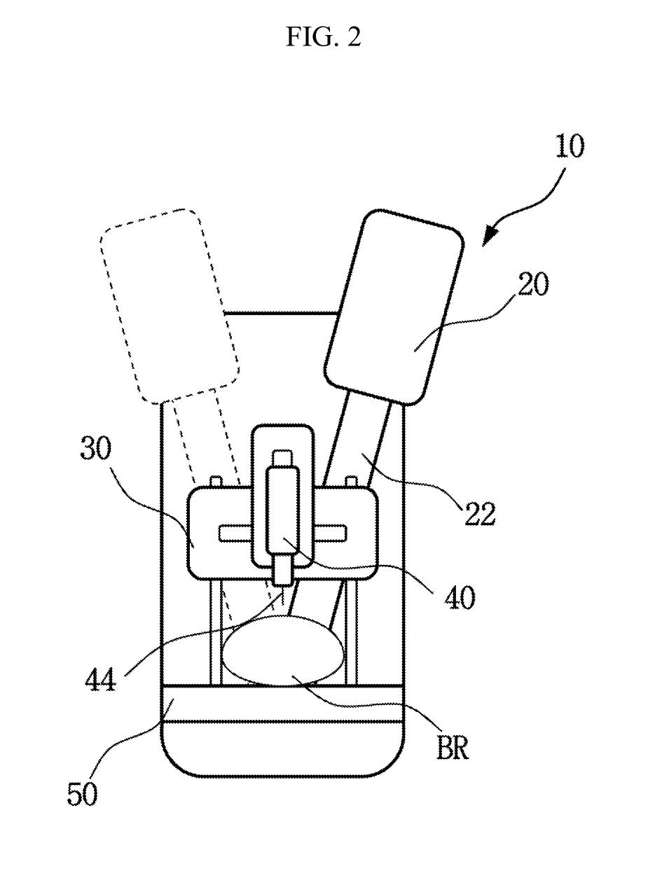

FIG. 3 is a front view illustrating a mammography apparatus according to a present invention;

FIG. 4 is a front view showing a biopsy needle guiding apparatus provided on the mammography apparatus of FIG. 3;

FIG. 5 is a front view showing a main body of the mammography apparatus according to the present invention so as to illustrate a stereotactic biopsy process; and

FIG. 6 is a front view illustrating another embodiment of a biopsy needle guiding apparatus provided on the mammography apparatus of FIG. 3.

### DESCRIPTION OF THE PREFERRED EMBODIMENTS

The following embodiments of the present invention are merely exemplary to easily explain the spirit of the present invention to those skilled in the art. Therefore, the present invention is not limited to the following embodiments, and various modifications are possible. In the drawings, the size of each element, the thickness of lines illustrating the element, etc. may be exaggeratedly expressed in the drawings for the sake of understanding the present invention. The same reference numerals are used throughout the different drawings to designate the same or similar components.

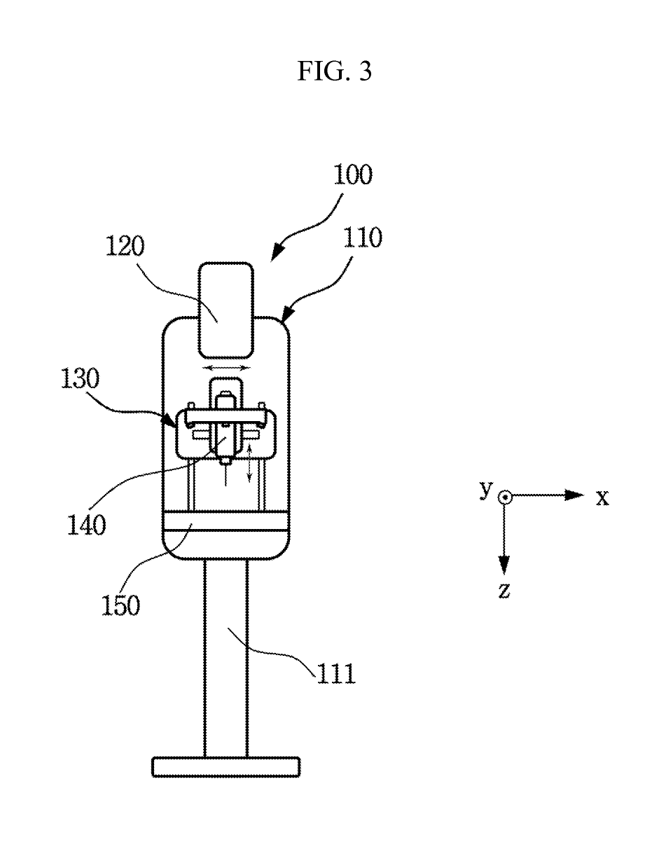

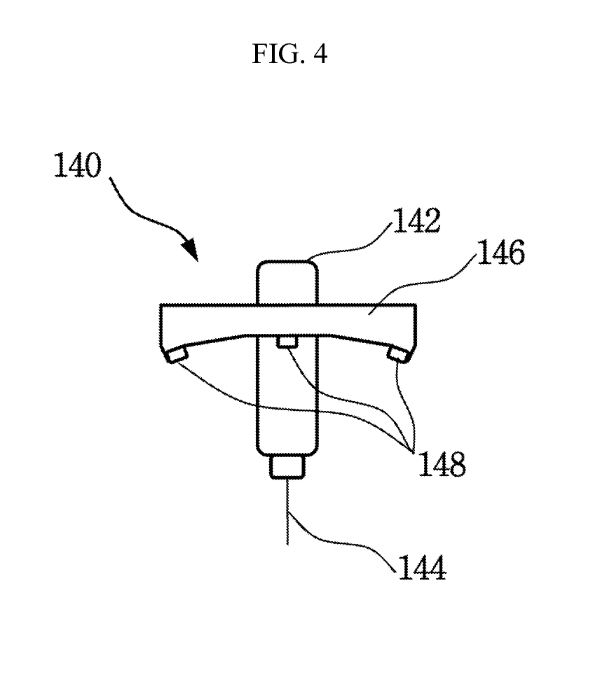

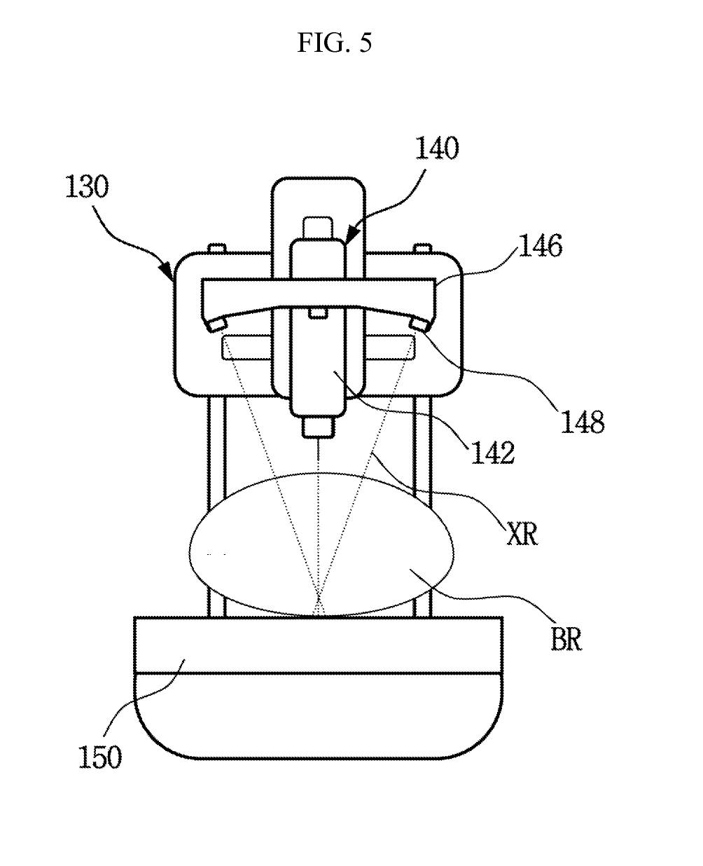

FIG. 3 is a front view showing a mammography apparatus which is an example of an imaging apparatus according to the present invention. FIG. 4 is a front view showing a biopsy needle guiding apparatus provided on the mammography apparatus of FIG. 3. FIG. 5 is a front view showing a main body of the mammography apparatus according to the present invention so as to illustrate a stereotactic biopsy process.

As shown in FIG. 3, the mammography apparatus 100 which is an example of the imaging apparatus according to the present invention includes a main body 110 which has basic components for X-ray imaging, and a support column 111 which guides the main body 110 so as to be vertically movable. The support column 111 has a vertical column shape and enables the main body 110 to elevate therealong.

The main body 110 has an arc shape in such a way that upper and lower ends thereof face each other. In other words, the main body 110 generally has a C shape or the like when viewed along the x-axis. A generator or a main X-ray source 120 which emits X-rays towards the lower end of the main body 110 is provided on the upper end of the main body 110. Facing the main X-ray source 120, a detector 150 is provided on the lower end of the main body 110 and receives X-rays from the main X-ray source 120. A needle guiding apparatus holder 130 is installed on main body 110 between the main X-ray source 120 and the detector 150. The biopsy needle guiding apparatus 140 is preferably removably provided on the needle guiding apparatus holder 130.

Provided on the upper end of the main body 110, the main X-ray source 120 generates X-rays in such a way as to collide electrons which have high kinetic energy with a metal target. Preferably, the main X-ray source 120 includes an optical system such as a collimator which controls the direction of X-ray radiation or the area of X-ray radiation.

The detector 150 which is provided on the lower end of the main body is a means for receiving an X-ray which passes through a target body BR such as the breast of a patient and obtaining an image. The detector 150 which allows the target body BR to be placed thereon also functions as a support for supporting the target body BR. In other word, after the target body BR is placed on the detector 150, the target body BR is imaged by the main X-ray source 120 and the detector 150.

Although it is not shown in the drawings, preferably, a pressure pad which presses the target body BR placed on the detector 150 is provided between the main X-ray source 120 of the main body 110 and the detector 150 so as to prevent an abnormal portion in the target body BR from being covered with breast tissue. The pressure pad can press the target body BR when the target body BR is imaged.

The detector 150 generates an electrical signal for each position in proportion to the incident amount of X-rays. The detector 150 reads an electrical signal and position information and processes them using an image processing algorithm, thus obtaining an X-ray image of the target body BR. Here, according to an X-ray conversion method, the detector 150 can be classified into a direct conversion type which directly obtains an electrical signal from an X-ray without a separate intermediate step, and an indirect conversion type which indirectly obtains an electrical signal from a visible ray that has been converted from an X-ray. Other well known technologies can also be used for the detector 150.

The needle guiding apparatus holder 130 holds and supports the biopsy needle guiding apparatus 140 that is a biopsy apparatus such that the biopsy needle guiding apparatus 140 is disposed between the main X-ray source 120 and the detector 150. The needle guiding apparatus holder 130 may be fixed to the main body 110 or, alternatively, it may be coupled to the main body 110 so as to be movable in a z-axis direction, as needed.

Preferably, the biopsy needle guiding apparatus 140 is removably coupled and supported to the needle guiding apparatus holder 130. The needle guiding apparatus holder 130 includes x-axis and y-axis stages which make it possible to move the biopsy needle guiding apparatus 140 in the x-axis and y-axis direction, and may optionally include an x-axis stage which makes it possible to move the biopsy needle guiding apparatus 140 in the x-axis direction.

The biopsy needle guiding apparatus 140 is a biopsy apparatus provided with a needle which is inserted into the target body BR to extract tissue of the target body BR. The biopsy needle guiding apparatus 140 is coupled to the needle guiding apparatus holder 130, preferably, so as to be removable. As shown in FIG. 4, the biopsy needle guiding apparatus 140 according to the present invention includes a needle guiding apparatus body 142 which vertically extends a predetermined length, a biopsy needle 144 which is provided under a lower end of the needle guiding apparatus body 142, an X-ray source mounting part 146 which is provided at a predetermined position on the needle guiding apparatus body 142 and horizontally extends a predetermined length, and three needle X-ray sources 148 which are mounted to the X-ray source mounting part 146. The three needle X-ray sources 148 are spaced apart from each other at regular intervals in a longitudinal direction of the X-ray source mounting part 146.

A general X-ray source includes a field emission cathode which emits electrons, and a target metal part that is an anode which collides with emitted electrons and creates an X-ray. The field emission cathode is a device which applies an electric field in a vacuum and emits electrons. In the conventional technique, a filament, etc. is used as the field emission cathode, but recently as nanotechnology has evolved, a nanoemitter such as a carbon nanotube is used as the filed emission cathode. Compared to the conventional X-ray source which uses the filament cathode, the X-ray source that uses the carbon nanotube has a reduced size and can solve a conventional problem of excessive power consumption caused by heating the filament cathode and overcome a limit in focusing electron beams. The configuration and function of the X-ray source are well known in the art related to the present invention, so that detailed description thereof will be omitted.

In the needle guiding apparatus 140 according to the present invention, because the carbon nanotube X-ray sources that have a reduced size are used, it is easy to install the three needle X-ray sources 148 in such a way that they are spaced apart from each other at regular intervals.

The needle X-ray source 148 that is in the middle of the three needle X-ray sources 148 (hereinafter, referred to as “the middle needle X-ray source 148”) is disposed on a line extending along the longitudinal axis of the biopsy needle 144. The other two needle X-ray sources 148 are disposed on the left and right sides of the middle needle X-ray source 148 at positions spaced apart therefrom by a predetermined distance. If a point of the detector 150, onto which an image is vertically projected from the middle X-ray source 148 when the biopsy needle guiding apparatus 140 is at a height of a standing-by position, refers to a reference point, the three needle X-ray sources 148 are arranged such that they face the reference point with the same distance from the reference point. In other words, the three needle X-ray sources 148 are arranged towards the reference point along a circular arc having the reference point as the center thereof. Preferably, the three needle X-ray sources 148 are spaced apart from each other at angular intervals ranging from 15 degrees to 30 degrees from the reference point.

Hereinafter, using the mammography apparatus 100 having the above-mentioned construction according to the present invention, a method of taking an X-ray image of the target body BR, for example, the breast, of the patient and extracting tissue of an abnormal portion will be explained.

When the patient is positioned in an imaging position of the mammography apparatus 100 standing up or sitting down, the main body 110 is moved upwards or downwards along the support column 111 and adjusted in position such that the target body BR is placed on the detector 150.

Thereafter, the main X-ray source 120 emits X-rays to the target body BR. The detector 150 which is disposed under the target body BR receives the X-rays that have been passed through the target body BR. The detector 150 generates an electrical signal for each position in proportion to the incident amount of the received X-rays. The detector 150 reads the electrical signal and position information and processes them with image processing algorithm, thus obtaining an X-ray image of the target body BR. If an abnormal portion is detected in the target body BR, a two-dimensional position of the abnormal portion in the x-y plane is obtained from the image obtained by the main X-ray source 120.

Subsequently, the X-axis and Y-axis stages of the needle guiding apparatus holder 130 are operated so that the biopsy needle guiding apparatus 140 is moved to the approximate position of the abnormal portion that has been obtained through the above-mentioned process. That is, the biopsy needle guiding apparatus 140 is disposed above the approximate position of the abnormal portion. Provided on the X-ray source mounting part 146 of the biopsy needle guiding apparatus 140, the three needle X-ray source 148 emits X-rays to the target body BR, in more detail, the abnormal portion BR, by turns (or at the same time). Disposed under the target body BR, the detector 150 receives the X-rays which have passed through the target body BR and obtains an X-ray image.

A three-dimensional position, that is, x-, y- and z-positions, of the abnormal portion can be obtained by trigonometry from the three X-ray images of the target body BR that contain the abnormal portion and the vicinity thereof. Based on the x- and y-position data of the abnormal portion obtained from the three needle X-ray sources 148, the x-axis and y-axis stages of the needle guiding apparatus holder 130 are precisely operated so that the biopsy needle guiding apparatus 140 is exactly disposed over the abnormal portion. That is, the biopsy needle guiding apparatus 140 is adjusted to the exact position. Finally, based on the z-position data of the abnormal portion obtained from the three needle X-ray sources 148, the biopsy needle guiding apparatus 140 or, in some cases, the needle guiding apparatus holder 130, moves the biopsy needle 144 forwards and backwards in the z-axis direction and extracts tissue of the abnormal portion.

As such, the biopsy needle guiding apparatus 140 is moved to the approximate position of the abnormal portion obtained from the main X-ray source 120, and the three needle X-ray sources 148 of the biopsy needle guiding apparatus 140 precisely image the abnormal portion once more to obtain the exact three-dimensional position of the abnormal portion. Therefore, even if the target body BR moves to some degree before the target body BR is imaged by the three needle X-ray sources 148 of the biopsy needle guiding apparatus 140 after the target body BR has been imaged by the main X-ray source 120, the measurement of the three-dimensional position of the abnormal portion tissue can be conducted without being impeded. Therefore, compared to the conventional technique, during the X-ray imaging process, the fatigue degree of the patient and inspector can be reduced.

Also, the mammography apparatus 100 according to the present invention has a simple structure having no separate drive unit for rotating the main X-ray source for stereotactic biopsy, thus facilitating the maintenance of the mammography apparatus, extending the lifetime of the apparatus, and reducing the overall size of the apparatus and the production cost thereof.

In the above-described embodiment, although the biopsy needle guiding apparatus 140 has been illustrated as including the three needle X-ray sources 148, the present invention may be embodied in such a way that the only two needle X-ray sources 148 are installed in the biopsy needle guiding apparatus 140 and the main X-ray source 120 which is installed on the main body 110 of the mammography apparatus 100 is used in lieu of the middle needle X-ray source 148. That is, the three-dimensional position of the abnormal portion of the target body BR may be measured by using the three X-ray sources configured such that the two needle X-ray sources 148 which are installed on the biopsy needle guiding apparatus 140 and the single main X-ray source 120 which is installed on the main body 110.

In this case, when the biopsy needle guiding apparatus 140 is moved to measure the three-dimensional position of the abnormal portion of the target body BR, the abnormal portion, one of the two needle X-ray sources 148 and the main X-ray source 120 must not be aligned with each other. Given this, as shown in FIG. 4, it is preferable that the middle X-ray source 148 of the three needle X-ray sources 148 which are installed in the biopsy needle guiding apparatus 140 be omitted.

Especially, when the biopsy needle guiding apparatus 140 is moved in the mammography apparatus 100, the needle X-ray sources 148 are moved along with the biopsy needle guiding apparatus 140, but the main X-ray source 120 is fixed. Therefore, when imaging the abnormal portion for measuring the three-dimensional position of the abnormal portion using the two the needle X-ray sources 148 and the main X-ray source 120, the position relationship between the X-ray sources must be taken into account. Moreover, because the distance from the abnormal portion of the target body BR to the needle X-ray source differs from the distance therefrom to the main X-ray source 120, compensation for this must also be taken into account.

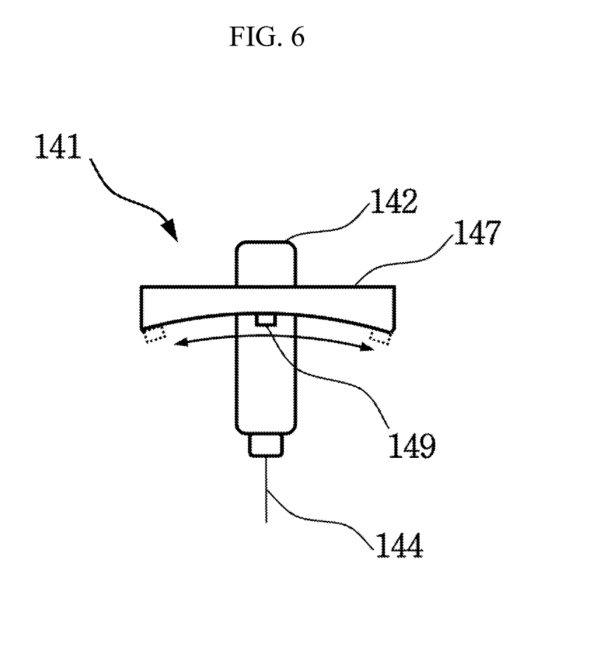

Meanwhile, the present invention may be embodied by using only a single needle X-ray source which is installed in the biopsy needle guiding apparatus 140 so as to be movable. For example, as shown in FIG. 6, a biopsy needle guiding apparatus 141 according to another embodiment of the present invention includes a needle guiding apparatus body 142 which is vertically extends a predetermined length, a biopsy needle 144 which is provided under the needle guiding apparatus body 142, an X-ray source mounting part 147 which is provided at a predetermined position on the needle guiding apparatus body 142 and horizontally extends a predetermined length, and a needle X-ray source 149 which is mounted to the X-ray source mounting part 147 so as to be movable in a longitudinal direction of the X-ray source mounting part 147. The needle X-ray source 149 can move in the direction designated by the arrow of FIG. 6 and image the target body BR, for example, at the three positions designated by the dotted lines and solid line.

The X-ray source mounting part 147, on which the needle X-ray source 149 is movably provided, preferably has a circular arc shape such that the needle X-ray source 149 faces the reference point described in FIG. 4 and moves along the X-ray source mounting part 147 with the constant distance between it and the reference point. Preferably, the three positions designated by the dotted lines and the solid line are positioned in such a way that the two positions designated by the dotted lines are spaced apart from the position designated by the solid line at angular intervals ranging from 15 degrees to 30 degrees on the reference point.

As described above, if the needle guiding apparatus 140 or 141 with one or more small X-ray sources according to the present invention is installed in the conventional mammography apparatus, it becomes possible to perform stereotactic biopsy even using the conventional mammography apparatus which has otherwise been unable to conduct stereotactic biopsy. As such, the applicability of the existing apparatus can be enhanced by the present invention. In other words, the needle guiding apparatus 140 or 141 according to the present invention can be applied to an inexpensive existing product only for screening, thereby making it possible for the existing product to compete with an expensive high-end product that can conduct stereotactic biopsy.

Although the preferred embodiments of the present invention have been disclosed for illustrative purposes, those skilled in the art will appreciate that various modifications, additions and substitutions are possible, without departing from the scope and spirit of the invention as disclosed in the accompanying claims. Therefore, the embodiment disclosed in this specification is only for illustrative purposes rather than limiting the technical spirit of the present invention. The scope of the present invention must be defined by the accompanying claims, and all technical spirits that are in the equivalent range to the claims must be regarded as falling within the scope of the present invention.

1. A biopsy needle guiding apparatus, comprising:
a needle guiding apparatus body provided with a biopsy needle; and
at least one X-ray source provided on the needle guiding apparatus body.

2. The biopsy needle guiding apparatus as set forth in claim 1, wherein the at least one X-ray source comprises one X-ray source provided so as to be movable in a direction crossing a longitudinal direction of the biopsy needle.

3. The biopsy needle guiding apparatus as set forth in claim 1, wherein the at least one X-ray source comprises two or more X-ray sources spaced apart from each other in a direction crossing a longitudinal direction of the biopsy needle.

4. The biopsy needle guiding apparatus as set forth in claim 3, wherein the X-ray sources are arranged at left and right symmetrical positions spaced apart from a point aligned with a longitudinal axis of the biopsy needle by a same distance within an angular range from 15 degrees to 30 degrees.

5. The biopsy needle guiding apparatus as set forth in claim 3, further comprising
an X-ray source disposed at a position aligned with the longitudinal axis of the biopsy needle.

6. An imaging apparatus, comprising:
a main X-ray source emitting X-rays to a target body;
a detector receiving the X-rays that have passed through the target body;
a main body supporting the X-ray source and the detector; and
a biopsy needle guiding apparatus having: a needle guiding apparatus body provided on the main body between the main X-ray source and the detector, the needle guiding apparatus body being provided with a biopsy needle; and at least one X-ray source provided on the needle guiding apparatus body.

7. The imaging apparatus as set forth in claim 6, wherein the biopsy needle guiding apparatus body is removably provided on the main body.

8. A biopsy method using an imaging apparatus comprising: a main X-ray source and a detector disposed facing each other with a target body therebetween; and a biopsy needle guiding apparatus disposed between the target body and the main X-ray source, the biopsy needle guiding apparatus comprising a biopsy needle and at least one X-ray source, the biopsy method comprising:
obtaining an X-ray transmission image of the target body and determining whether the target body is normal or abnormal; and
calculating a three-dimensional position of an abnormal portion of the target body using the needle X-ray source and the detector, and extracting tissue of the abnormal portion using the biopsy needle.

9. The biopsy needle guiding apparatus as set forth in claim 4, further comprising
an X-ray source disposed at a position aligned with the longitudinal axis of the biopsy needle.
